(19) United States
(12) Patent Application Publication
(10) Pub. No.: US 2009/0093461 A1
(43) Pub. Date: Apr. 9, 2009

(54) Methods of Treating Anxiety and Mood Disorders

(75) Inventors: Patricia C. Davis, Wilmington, DE (US); Jeffrey Goldstein, Wilmington, DE (US); Scott W. Grimm, Wilmington, DE (US); Raymond F. Suckow, West Paterson, NJ (US); Helen R. Winter, Mystic, CT (US)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/139,095

(22) Filed: Jun. 13, 2008

Related U.S. Application Data
(63) Continuation-in-part of application No. 11/327,639, filed on Jan. 6, 2006.
Application No. 11/327,639 is a continuation-in-part of application No. 10/883,024, filed on Jul. 1, 2004, now abandoned.
(60) Provisional application No. 60/737,884, filed on Nov. 18, 2005.
(60) Provisional application No. 60/642,262, filed on Jan. 7, 2005.
(60) Provisional application No. 60/484,365, filed on Jul. 2, 2003.

(51) Int. Cl. A61K 31/554; A61P 25/22

(52) U.S. Cl. 514/211.13

(57) ABSTRACT

Methods of treating at least one symptom or condition associated with Anxiety Disorders and/or Mood Disorders comprising administering an effective amount of a compound of Formula I

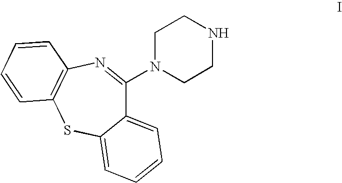

or its pharmaceutically acceptable salt are provided herein.

### CROSS-REFERENCE TO RELATED APPLICATIONS

This application is a continuation-in-part of: 1) U.S. Ser. No. 11/327,639 filed Jan. 6, 2006, which claims priority to U.S. provisional application Ser. No. 60/737,884 filed Nov. 18, 2005 and to U.S. provisional application Ser. No. 60/642,262 filed Jan. 7, 2005, and which is a continuation-in-part of U.S. Ser. No. 10/883,024 filed Jul. 1, 2004, which claims priority to U.S. provisional application Ser. No. 60/484,365 filed Jul. 2, 2003; and 2) U.S. Ser. No. 11/327,636 filed Jan. 6, 2006, which claims priority to U.S. provisional application Ser. No. 60/737,860, filed Nov. 18, 2005, and to U.S. provisional application Ser. No. 60/642,262 filed Jan. 7, 2005, and which is a continuation-in-part of U.S. Ser. No. 10/883,024 filed Jul. 1, 2004, which claims priority to U.S. provisional application Ser. No. 60/484,365 filed Jul. 2, 2003; each which is incorporated herein by reference in its entirety.

### FIELD OF THE INVENTION

The present invention provides pharmaceutical compositions and methods relating to 11-piperazin-1-yldibenzo[b,f][1,4]thiazepine.

### BACKGROUND OF THE INVENTION

A goal of antipsychotic drug development has been to develop agents with increased efficacy and safety along with fewer of the side effects commonly associated with the older antipsychotic medications. Quetiapine fumarate is described in U.S. Pat. No. 4,879,288, which is incorporated herein by reference. Quetiapine fumarate is able to treat both the positive (hallucinations, delusions) and negative symptoms (emotional withdrawal, apathy) of psychosis and is associated with fewer neurological and endocrine related side effects compared to older agents. Quetiapine fumarate has also been associated with a reduction in hostility and aggression. Quetiapine fumarate is associated with fewer side effects such as EPS, acute dystonia, acute dyskinesia, as well as tardive dyskinesia. Quetiapine fumarate has also helped to enhance patient compliance with treatment, ability to function and overall quality of life, while reducing recidivism. P. Weiden et al., Atypical antipsychotic drugs and long-term outcome in schizophrenia, 11 J. Clin. Psychiatry, 53-60, 57 (1996). Because of quetiapine fumarate's enhanced tolerability profile its use is particularly advantageous in the treatment of patients that are hypersensitive to the adverse effects of antipsychotics (such as elderly patients).

Derivatives of 11-(piperazin-1-yl)dibenzo[b,f][1,4]-thiazepines and related compounds including metabolites of quetiapine were prepared and evaluated in E. Warawa et al. Behavioral approach to nondyskinetic dopamine antagonists: identification of Seroquel, 44, J. Med. Chem., 372-389 (2001). Quetiapine metabolism has been reported in C. L. Devane et al. Clin. Pharmacokinet., 40(7), 509-522 (2001) wherein the structure of 11-piperazin-1-yldibenzo[b,f][1,4]thiazepine (see Formula I below) was shown in FIG. 1. This compound was reported by Schmutz et al. in U.S. Pat. No: 3,539,573. This compound has also been used in processes for preparing quetiapine as reported in U.S. Pat. No. 4,879,288. It has now been found that 11-piperazin-1-yldibenzo[b,f][1,4]thiazepine is a circulating metabolite of quetiapine in humans.

### SUMMARY OF THE INVENTION

11-Piperazin-1-yldibenzo[b,f][1,4]thiazepine has the structure as shown by Formula I:

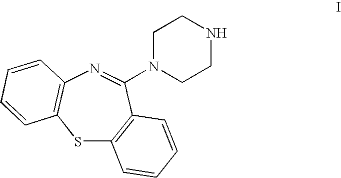

Provided herein is a method of treating, comprising the administration of an effective amount of Formula I or its pharmaceutically acceptable salt to a mammal, at least one symptom or condition associated with but not limited to: 1) Anxiety Disorders including, but not limited to, Panic Disorder Without Agoraphobia, Panic Disorder With Agoraphobia, Agoraphobia Without History of Panic Disorder, Specific Phobia, Social Phobia, Obsessive-Compulsive Disorder, Post-traumatic Stress Disorder, Acute Stress Disorder, Generalized Anxiety Disorder and Generalized Anxiety Disorder Due to a General Medical Condition; and 2) Mood Disorders including, but not limited to, a) Depressive Disorders including, but not limited to, Major Depressive Disorder and Dysthymic Disorder, and b) Bipolar Depression and/or Bipolar mania including, but not limited to, Bipolar I Disorder including, but not limited to, those with manic, depressive or mixed episodes, and Bipolar II Disorder, c) Cyclothymic Disorder, and d) Mood Disorder Due to a General Medical Condition. Examples of definitions of the above conditions and disorders can be found, for example, in the American Psychiatric Association: Diagnostic and Statistical Manual of Mental Disorders, Fourth Edition, Text Revision, Washington, D.C., American Psychiatric Association, 2000.

In another aspect of the invention provided is a pharmaceutical composition comprising an effective amount of the compound of Formula I or its pharmaceutically acceptable salt and at least one pharmaceutically acceptable carrier. Also provided is a method of treating the symptoms or conditions provided herein comprising administering to a mammal a pharmaceutical composition described above. Also provided is the use of the compound of Formula I or its pharmaceutically acceptable salt and/or the above-mentioned pharmaceutical composition in the treatment of the symptoms or conditions provided herein in mammals. Also provided is the use of the compound of Formula I or its pharmaceutically acceptable salt administered in combination with one or more other therapeutically active agents. Further, provided herein is the use of the compound of Formula I or its pharmaceutically acceptable salt and/or the pharmaceutical composition in the manufacture of a medicament for use in the treatment of the symptoms or conditions provided herein in mammals.

### BRIEF DESCRIPTION OF THE DRAWINGS

FIG. 1 depicts an X-ray powder diffraction (XRPD) pattern consistent with crystalline 11-piperazin-1-yldibenzo[b,f][1,4]thiazepine having Form A.

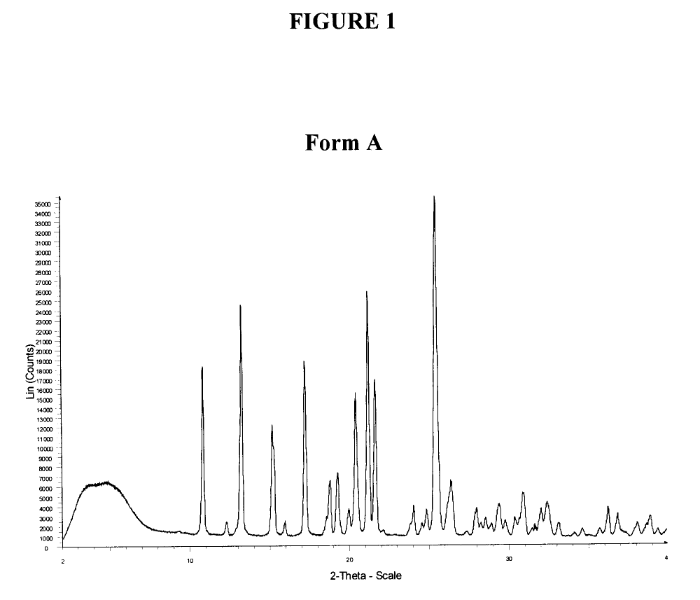

FIG. 2 depicts antidepressant effects of 11-piperazin-1-yldibenzo[b,f][1,4]thiazepine in the mouse forced swim test.

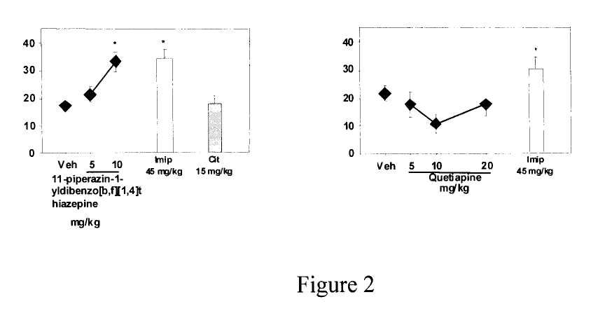

### DETAILED DESCRIPTION OF THE INVENTION

The compound of Formula I is a dibenzothiazepine that has been shown to have 5HT_{1A} partial agonist activity and has shown in vivo efficacy in an animal model for anxiety. Preliminary positron emission topography (PET) scans of primate subjects showed that the compound of Formula I reaches the brain and occupies D1, D₂, 5-HT_{2A}, and 5-HT_{1A} receptors and the 5HT Transporter. The compound of Formula I also has shown antidopaminergic activity. It has been shown to interact with a broad range of neurotransmitter receptors but has a higher affinity for serotonin (5-HT₂) receptors relative to dopamine (D₂) receptors in the brain. However, the compound of Formula I was not shown to be efficacious in a mouse standard apomorphine swim test (p.o.) and in a rat D-Ampehtamine locomotor activity test (s.c.). The compound of Formula I has also been shown to have partial 5HT_{1A} agonist activity and has shown in vivo efficacy in mouse and rat models for depression. The compound of Formula I may be used as an antipsychotic with a reduction in the potential to cause side effects such as acute dystonia, acute dyskinesia, as well as tardive dyskinesia typically seen with antipsychotics. Results generated from alpha receptor binding data for 11-piperazin-1-yldibenzo[b,f][1,4]thiazepine suggest that the compound of Formula I will have improved tolerability over that of quetiapine and suggest that one would observe a reduced incidence of hypotension. Further the compound of Formula I may be used to treat patients of all ages and may be advantageous in the treatment of elderly patients.

The term “mammal” means a warm-blooded animal, preferably a human.

The compound of Formula I may be made by a variety of methods known in the chemical arts. The compound of Formula I may be prepared by starting from known compounds or readily prepared intermediates including taking the lactam of Formula II:

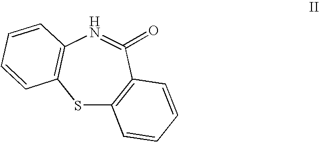

which may be prepared by methods well known in the literature, for example, as described by J. Schmutz et al. Helv. Chim. Acta., 48:336 (1965). The lactam of Formula II is treated with phosphorus chloride to generate the immino chloride of Formula III:

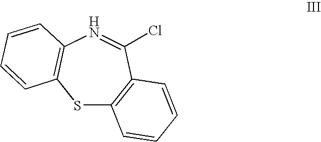

The immino chloride of Formula III may also be generated with other agents such as thionyl chloride or phosphorous pentachloride. The imino chloride is then reacted with piperazine to give the compound of Formula I.

The compound of Formula I provided herein is useful as a free base, but may also be provided in the form of a pharmaceutically acceptable salt, and/or in the form of a pharmaceutically acceptable solvate (including hydrates). For example, pharmaceutically acceptable salts of Formula I include those derived from mineral acids such as for example: hydrochloric acid, nitric acid, phosphoric acid, sulfuric acid, hydroiodic acid, nitrous acid, and phosphorous acid. Pharmaceutically acceptable salts may also be developed with organic acids including aliphatic mono dicarboxylates and aromatic acids. Other pharmaceutically acceptable salts of Formula I include, but are not limited to, hydrochloride, sulfate, pyrosulfate, bisulfate, bisulfite, nitrate, and phosphate.

A clinician may determine the effective amount by using numerous methods already known in the art. The term “treating” within the context of the present invention encompasses to administer an effective amount of the compound of the present invention, to mitigate either a pre-existing disease state, acute or chronic, or a recurring symptom or condition. This definition also encompasses prophylactic therapies for prevention of recurring conditions and continued therapy for chronic disorders.

A particular amount of the compound of Formula I or its pharmaceutically acceptable salt can be administered in an amount up to about 750 mg per day; particularly from about 75 mg to about 750 mg per day. In another particular aspect of the invention the amount of the compound of Formula I, or its pharmaceutically acceptable salt, may be administered from about 1 mg to about 600 mg per day. In another aspect of the invention the compound of Formula I or its pharmaceutically acceptable salt may be administered from about 100 mg to about 400 mg per day.

The compound of Formula I or its pharmaceutically acceptable salt may be administered comprising a predetermined dosage of the compound of Formula I to a mammal between one and four times a day, wherein the predetermined dosage is from about 1 mg to about 600 mg.

The present invention also provides a method of treating the symptoms or conditions provided herein comprising the step of administering an initial predetermined dosage of a compound of Formula I to a human patient twice a day, wherein the predetermined dosage is between 1 mg and 30 mg with increases in increments of 1-50 mg twice daily on the second and third day as tolerated. Thereafter, further dosage adjustments can be made at intervals of no less than 2 days.

In one embodiment of the invention the pharmaceutical composition comprises up to about 750 mg of the compound of Formula I or its pharmaceutically acceptable salt, particularly from about 75 mg to about 750 mg.

In another embodiment of the invention, the pharmaceutical composition may comprise from about 1 mg to about 600 mg of the compound of Formula I or a pharmaceutically acceptable salt thereof.

In another embodiment of the invention, the pharmaceutical composition may comprise from about 100 mg to about 400 mg of the compound of Formula I or a pharmaceutically acceptable salt thereof.

The pharmaceutical composition of the invention may accordingly be obtained by conventional procedures using conventional pharmaceutical excipients. Thus, pharmaceutical compositions intended for oral use may contain, for example, one or more coloring, sweetening, flavoring and/or preservative agents.

For preparing pharmaceutical compositions from the compound of Formula I of this invention, inert, pharmaceutically acceptable carriers can be either solid or liquid. Solid form preparations include powders, tablets, dispersible granules, capsules, cachets, and suppositories.

The composition of the invention may be administered by any route including orally, intramuscularly, subcutaneously, topically, intranasally, intraperitoneally, intrathoracially, intravenously, epidurally, intrathecally, intracerebroventricularly and by injection into the joints.

The amount of active ingredient that is combined with one or more excipients to produce a single dosage form, such as an oral dosage form, will necessarily vary depending upon the host treated and the particular route of administration. The size of the dose for therapeutic or prophylactic purposes of a compound of the Formula I will naturally vary according to the nature and severity of the symptoms or conditions, the age and sex of the animal or patient and the route of administration, according to well known principles of medicine.

Another aspect of the invention provides a compound of Formula I, or its pharmaceutically acceptable salt or solvate thereof, for use in treating the symptoms or conditions provided herein.

In a further aspect, the present invention provides the use of a compound of Formula I, or a pharmaceutically acceptable salt or solvate thereof, in the manufacture of a medicament for use in treating the symptoms or conditions provided herein.

In a further aspect, the present invention relates to methods of treating at least one of the above described symptoms or conditions comprising administering to a mammal an effective amount of the compound of Formula I or its pharmaceutically acceptable salt and one or more of other therapeutically active agents, benzodiazepines, 5-HT_{1A} ligands, 5-HT_{1B} ligands, 5-HT_{1D} ligands, mGluR2A agonists, mGluR5 antagonists, antipsychotics, NK1 receptor antagonists, antidepressants, serotonin reuptake inhibitors, GABA II ligands, or mood stabilizers administered in combination as part of the same pharmaceutical composition, as well as to methods in which such active agents are administered separately as part of an appropriate dose regimen designed to obtain the benefits of combination therapy.

The appropriate dose regimen, the amount of each dose of an active agent administered, and the specific intervals between doses of each active agent will depend upon the subject being treated, the specific active agent being administered and the nature and severity of the specific disorder or condition being treated. In general, the compounds of this invention, when used as either a single active agent or when used in combination with another active agent, will be administered to a subject in an amount up to about 750 mg per day, in single or divided doses. Such compounds may be administered on a regimen of up to 6 times per day, preferably 1 to 4 times per day. Variations may nevertheless occur depending upon the subject being treated and the individual response to the treatment, as well as on the type of pharmaceutical formulation chosen and the time period and interval at which such administration is carried out. In some instances, dosage levels below the lower limit of the aforesaid range may be more than adequate, while in other cases larger doses may be employed to achieve the desired effect, provided that such larger doses are first divided into several small doses for administration throughout the day.

Exemplary benzodiazepines may include but are not limited to adinazolam, alprazolam, bromazepam, clonazepam, chlorazepate, chlordiazepoxide, diazepam, estazolam, flurazepam, balezepam, lorazepam, midazolam, nitrazepam, oxazepam, quazepam, temazepam, triazolam and equivalents thereof.

Exemplary 5-HT_{1A} and/or 5HT_{1B} ligands may include but are not limited to buspirone, alnespirone, elzasonan, ipsapirone, gepirone, zopiclone and equivalents thereof.

Exemplary mGluR 2 agonists may include (1S,3R)-1-aminocyclopentane-1,3-dicarboxylic acid, (2S,3S,4S)alpha-(carboxycyclopropyl)glycine, and 3,5-dihydroxyphenylglycine.

Exemplary antidepressants may include but are not limited to maprotiline, amitriptyline, clomipramine, desipramine, doxepin, imipramine, nortryptyline, protriptyline, trimipramine, SSRIs and SNRIs such as fluoxetine, paroxetine, citalopram, escitalopram, sertraline, venlafaxine, fluoxamine, and reboxetine.

Exemplary antipsychotics may include but are not limited to clozapine, risperidone, quetiapine, olanzapine, amisulpride, sulpiride, zotepine, chlorpromazine, haloperidol, ziprasidone, and sertindole.

Exemplary mood stabilizers may include but are not limited to Valproic acid (valproate) and its derivative (e.g. divalproex), lamotrigine, lithium, verapamil, carbamazepine and gabapentin.

The following examples provided are not meant to limit the invention in any manner and are intended for illustrative purposes only.

### EXAMPLES

### Example 1

### Preparation of 11-piperazin-1-yldibenzo[b,f][1,4]thiazepine

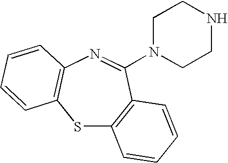

Into a 1000 ml round-bottom flask equipped with a magnetic stirring bar and reflux condenser with a nitrogen inlet was charged with 25.0 grams (g) (0.110 mole) of dibenzo[b,f][1,4]thiazepine-11(10-H)-one (made by the method disclosed by J. Schmutz et al. Helv. Chim. Acta., 48: 336 (1965)), as a dry solid, followed by 310 ml POCl₃ and 3 ml of N,N-dimethylaniline. The reaction mixture was heated at reflux (106° C.) for 6 hours giving a clear orange solution. The reaction was then cooled to room temperature, and POCl₃ removed on the rotary evaporator leaving an orange oil. This residue was partitioned between ice-water (500 ml) and ethyl acetate (800 ml). The layers were separated and the aqueous phase extracted with ethyl acetate (3×200 ml). The combined ethyl acetate extracts were dried over MgSO₄, filtered, and then stripped down on the rotary evaporator, leaving the crude imino chloride as a light yellow solid (26.26 g, 97% yield). The structure was confirmed by NMR and Mass Spectrum (300 MHz, CDCl₃; ES+, M+1=246.7). Crude imino chloride (27.35 g, 0.111 mole) was added to 1000 ml o-xylene in a 2000 ml round-bottom flask equipped with a magnetic stir bar and a reflux condenser with nitrogen inlet. To this solution was added commercially available piperazine (47.95 g, 0.557 mole) in one portion as a dry solid at room temperature. The mixture was stirred until nearly all the piperazine dissolved. Then the reaction mixture was heated at reflux (142° C.) for 40 hours (out of convenience). The reaction was then allowed to cool to room temperature, and an aliquot was partitioned between 1N NaOH/CH₂Cl₂. The organic phase was checked by TLC (silica gel, CH₂Cl₂/methanol 90:10, iodoplatinate visualized) and showed clean conversion to one major product (Rf=0.45). A drop of the reaction solution was diluted with CH₃CN to prepare a sample for LC/MS analysis, which confirmed the presence of the desired product (M+1=296.4). The reaction mixture was stripped down on the rotary evaporator under high vacuum to remove the xylene. The residue was partitioned between 1N NaOH (400 ml) and CH₂Cl₂ (200 ml). The layers were separated, and the aqueous phase further extracted with CH₂Cl₂ (3×200 ml). The combined CH₂Cl₂ extracts were washed with brine (200 ml), then dried over MgSO₄, filtered, and stripped down on the rotary evaporator to give the crude title compound as a yellow gum (35.3 g). The crude free base was purified by flash column chromatography over silica gel (600 g) eluting with a gradient of 0 to 20% Methanol in CH₂Cl₂. Fractions containing the pure desired product were combined and stripped down on the rotary evaporator, to afford the purified free base as a light yellow foam (25.67 g, 78% yield).

### Example 2

### Preparation of 11-piperazin-1-yldibenzo[b,f][1,4]thiazepine, dihydrochloride salt

The free base was converted to its dihydrochloride salt by dissolving it in a mixture of methanol (125 ml) and diethyl ether (125 ml), then treating with 250 ml of 1.0 M HCl/ether (Aldrich). An off-white gummy solid separated initially, and the mixture was further diluted with 500 ml ether. The gummy solid did not solidify on prolonged stirring. The solvents were decanted away from the gum. The gum was treated with absolute ethanol (200 ml), then stirred until crystallization occurred, giving a thick white suspension of crystals. This mixture was then slowly diluted with ether (800 ml) and allowed to stir overnight to complete the crystallization. The crystalline dihydrochloride salt was isolated by filtration, washed with ether (3×50 ml), then dried in vacuum at 60° C. to afford the dihydrochloride salt of the title compound as a white crystalline solid (31.64 g, 98.8% conversion).

### Analysis:

The product was characterized by NMR and LC/MS (300 MHz, CDCl₃; AP+, M+1=296.4).

### Example 3

### Preparation of crystalline 11-piperazin-1-yldibenzo[b,f][1,4]thiazepine

### Preparation A

Aqueous solution (584 ml; e.g., prepared by extraction of 11-piperazin-1-yldibenzo[b,f][1,4]thiazepine into water/HCl from a toluene solution such as described below in Preparation B) containing 11-piperazin-1-yldibenzo[b,f][1,4]thiazepine hydrochloride was charged to a jacketed 1 L flask. The flask was then charged with toluene (500 ml) and sodium hydroxide (48% w/w, 33.0 g). The mixture was stirred at 70° C. for 30 minutes and became white and cloudy. The mixture was then allowed to settle for 30 minutes and the phases were separated. The toluene layer was washed at 70° C. with 2×100 ml of water (1^{st} wash=pH 10.3; 2^{nd} wash=pH 8.0). The final toluene volume was 560 ml containing about 74 g of 11-piperazin-1-yldibenzo[b,f][1,4]thiazepine in good purity.

The above procedure was repeated for an additional four aqueous solutions of 11-piperazin-1-yldibenzo[b,f][1,4]thiazepine hydrochloride and the five resulting toluene solutions were combined and evaporated to dryness on a rotary evaporator. The resulting hard solid was then charged to a jacketed vessel and slurried with methyl-t-butyl ether (MTBE) (500 ml). The resulting slurry was stirred overnight at ambient temperature and then cooled to 5° C. and held for 4 hours. The solid 11-piperazin-1-yldibenzo[b,f][1,4]thiazepine product was isolated on a no. 3 sinter and washed with 200 ml of cold MTBE. The cake was dried in a vacuum oven overnight at 60° C. yielding 373 g of product.

### Preparation B

A toluene solution of 11-piperazin-1-yldibenzo[b,f][1,4]thiazepine (1500 ml, 0.686 mol) prepared by reaction of piperazine with 11-chloro-dibenzo[b,f][1,4]-thiazepine in toluene (see, e.g., U. S. Pat. No. 4,879,288) was treated with 1500 ml deionized water and 90 ml of HCl (32% w/w). The resulting mixture was heated to 70° C. and agitated for 45 minutes. Agitation was ceased and the mixture allowed to settle and phase separate for 30 minutes. The lower aqueous phase, containing the HCl salt of 11-piperazin-1-yldibenzo[b,f][1,4]thiazepine was isolated. The aqueous phase was then treated with 1000 ml of toluene and 99 g of aqueous NaOH (47% w/w). The resulting mixture was heated to 70° C. and agitated for 45 minutes. Agitation was ceased and the mixture allowed to settle and phase separate for 30 minutes. The lower aqueous phase was discarded and the upper organic phase retained to which 300 ml of deionized water was added. The resulting mixture was agitated for 15 minutes and then allowed to settle for 30 minutes. The aqueous phase was discarded and the organic phase retained. The organic phase was extracted once more with 300 ml of deionized water. About 750 ml of toluene from the organic phase was distilled out. The resulting concentrate was cooled to 60° C., then 200 ml of methyl-t-butyl ether (MTBE) was added. The resulting mixture was cooled to ambient temperature then seeded with Form A seed crystals. The seeded mixture was then cooled to 10° C. and held at this temperature for 3 hours under slow agitation. The resulting solid was isolated under suction via a no. 3 sinter. The solid product was then washed with 120 ml of MTBE at ambient temperature and dried at 40° C. under vacuum resulting in 175 g (86.4%) of crystalline product. Assay 99.9% w/w by HPLC area %.

Solid 11-piperazin-1-yldibenzo[b,f][1,4]thiazepine (30 g, 0.1016 mol) prepared as described above was slurried in isopropanol (120 ml). The resulting mixture was warmed to about 63-64° C. to completely dissolve the solid. The resulting solution was filtered through a preheated (about 55° C.) split Buchner funnel fitted with filter paper with a pore size of 6 μm. The filtered solution was then adjusted to 55° C. and seeded with seed crystals of Form A (0.024 g). The seeded solution was maintained at 55° C. for about 2 hours then linearly cooled to 40° C. over the course of 6 hours, linearly cooled to 20° C. over the course of 2 hours, and then linearly cooled to 0° C. over the course of 1 hour. The resulting slurry was held at 0° C. for 12 hours and the solid product cake (13 mm high×68 mm diameter) was isolated by filtration. The product cake was displacement washed with 30 ml isopropanol pre-chilled to 0° C. and the cake allowed to deliquor. The product was then dried at 40° C. under vacuum yielding 24.9 g (83%) of Form A. Assay by NMR: 98.9% w/w.

X-ray powder diffraction (XRPD) peak data of crystalline Form A is provided below in Chart A. The following instrument setting were used.

| | |
|---|---|
| Instrument | Bruker D8 Discover |
| Scan range | 2-40° 2θ |
| Step size | 0.007° 2θ |
| Scan speed | 0.2 sec/step |
| Scan type | 2TH/T |
| Lamp intensity | 35 kV/45 mA |

CHART A
(Form A)

| Angle | Intensity | Intensity |
|---|---|---|
| 2-Theta ° | Count | % |
| 10.8 | 18321 | 51.4 |
| 12.3 | 2390 | 6.7 |
| 13.3 | 24555 | 68.9 |
| 15.2 | 12193 | 34.2 |
| 15.3 | 9799 | 27.5 |
| 16.0 | 2414 | 6.8 |
| 17.2 | 18803 | 52.7 |
| 18.8 | 6502 | 18.2 |
| 19.3 | 7290 | 20.4 |
| 20.0 | 3666 | 10.3 |
| 20.4 | 15535 | 43.6 |
| 21.2 | 25874 | 72.6 |
| 21.7 | 16902 | 47.4 |
| 22.1 | 1473 | 4.1 |
| 24.1 | 3968 | 11.1 |
| 24.2 | 2197 | 6.2 |
| 24.9 | 3579 | 10 |
| 25.5 | 35663 | 100 |
| 26.4 | 6298 | 17.7 |
| 27.9 | 3290 | 9.2 |
| 28.0 | 3746 | 10.5 |
| 28.3 | 2206 | 6.2 |
| 28.6 | 2711 | 7.6 |
| 28.9 | 2142 | 6 |
| 29.4 | 4006 | 11.2 |
| 29.8 | 2464 | 6.9 |
| 30.4 | 2754 | 7.7 |
| 30.9 | 5213 | 14.6 |
| 31.0 | 5143 | 14.4 |
| 31.6 | 2053 | 5.8 |
| 32.1 | 3643 | 10.2 |
| 32.4 | 4234 | 11.9 |
| 32.5 | 3827 | 10.7 |
| 33.2 | 2102 | 5.9 |
| 34.6 | 1540 | 4.3 |
| 35.8 | 1543 | 4.3 |
| 36.3 | 3768 | 10.6 |
| 36.9 | 3086 | 8.7 |
| 38.1 | 2062 | 5.8 |
| 39.0 | 2801 | 7.9 |
| 39.4 | 1492 | 4.2 |

### Example 4

### α1 and α2 Receptor Profile

Differentiation of 11-piperazin-1-yldibenzo[b,f][1,4]thiazepine from quetiapine is based on alpha receptor binding data shown below.

| Receptor | Quetiapine Affinity (nM) | (I) Affinity (nM) |
|---|---|---|
| α1A | 22 | 108 |
| α1B | 39 | 75 |
| α1D | — | 185 |
| α2C | 28.9 | 820 |

The above affinity values were derived from the below results, methods and criteria.

| PRIMARY | | | | | | | | |
|---|---|---|---|---|---|---|---|---|
| BIOCHEMICAL ASSAY | SPECIES | CONC. | % INH. | IC₅₀ | | ki | | n |
| Adrenergic α_{1A} | rat | 0.3 μM | 51 | 0.268 ± 0.012 | μM | 0.108 ± 0.005 | μM | 1.03 ± 0.089 |
| Adrenergic α_{1B} | rat | 0.1 μM | 50 | 0.136 ± 0.0154 | | 0.075 ± 0.009 | μM | 1.1 ± 0.024 |
| Adrenergic α_{1D} | hum | 0.3 μM | 53 | 0.377 ± 0.049 | μM | 0.185 ± 0.024 | μM | 0.984 ± 0.111 |
| Adrenergic α_{2A} | hum | 3 μM | 51 | 2.82 ± 0.275 | μM | 1.06 ± 0.103 | μM | 0.969 ± 0.012 |
| Adrenergic α_{2B} | hum | 1 μM | 62 | 0.451 ± 0.097 | μM | 0.206 ± 0.0445 | μM | 0.902 ± 0.055 |
| Adrenergic α_{2C} | hum | 10 μM | 58 | 5.64 ± 1.01 | μM | 0.82 ± 0.146 | μM | 1.1 ± 0.079 |
| Adrenergic α_{1*} | rat | 0.1 μM | 61 | 0.0693 | μM | 0.0372 | μM | 0.964 |
| Adrenergic α_{2*} | rat | 10 μM | 73 | 1.41 | μM | 1.29 | μM | 0.592 |

Receptor binding methods, α-adrenergic subtype specific, are provided below.

203100 Adrenergic α_{1A}

| | |
|---|---|
| Source: | Wistar Rat submaxillary gland |
| Ligand: | 0.25 nM [³H] Prazosin |
| Vehicle: | 1% DMSO |
| Incubation Time/Temp: | 60 minutes @ 25° C. |
| Incubation Buffer: | 20 mM Tris-HCl, 0.5 mM EDTA, pH 7.4 |
| Non-Specific Ligand: | 10 μM Phentolamine |
| K_{D}: | 0.17 nM* |
| B_{MAX}: | 0.18 pmole/mg Protein* |
| Specific Binding: | 90%* |
| Quantitation Method: | Radioligand Binding |
| Significance Criteria: | ≧50% of max stimulation or inhibition |

203200 Adrenergic α_{1B}

| | |
|---|---|
| Source: | Wistar Rat liver |
| Ligand: | 0.25 nM [³H] Prazosin |
| Vehicle: | 1% DMSO |
| Incubation Time/Temp: | 60 minutes @ 25° C. |
| Incubation Buffer: | 20 mM Tris-HCl, 0.5 mM EDTA, pH 7.4 |
| Non-Specific Ligand: | 10 μM Phentolamine |
| K_{D}: | 0.31 nM* |
| B_{MAX}: | 0.18 pmole/mg Protein* |
| Specific Binding: | 90%* |
| Quantitation Method: | Radioligand Binding |
| Significance Criteria: | ≧50% of max stimulation or inhibition |

203400 Adrenergic α_{1D}

| | |
|---|---|
| Source: | Human recombinant HEK-293 cells |
| Ligand: | 0.6 nM [³H] Prazosin |
| Vehicle: | 1% DMSO |
| Incubation Time/Temp: | 60 minutes @ 25° C. |
| Incubation Buffer: | 50 mM Tris-HCl |
| Non-Specific Ligand: | 10 μM Phentolamine |
| K_{D}: | 0.58 nM* |
| B_{MAX}: | 0.17 pmole/mg Protein* |
| Specific Binding: | 80%* |
| Quantitation Method: | Radioligand Binding |
| Significance Criteria: | ≧50% of max stimulation or inhibition |

Receptor binding methods, α-adrenergic nonselective, are provided below.

203500 Adrenergic α_{1*} Non-Selective

| | |
|---|---|
| Source: | Wistar Rat brain |
| Ligand: | 0.25 nM [³H] Prazosin |
| Vehicle: | 1% DMSO |
| Incubation Time/Temp: | 30 minutes @ 25° C. |
| Incubation Buffer: | 50 mM Tris-HCl, 0.1% ascorbic acid |
| | 10 μM pargyline |
| Non-Specific Ligand: | 0.1 μM Prazosin |
| K_{D}: | 0.29 nM* |
| B_{MAX}: | 0.095 pmole/mg Protein* |
| Specific Binding: | 90%* |
| Quantitation Method: | Radioligand Binding |
| Significance Criteria: | ≧50% of max stimulation or inhibition |

203900 Adrenergic α_{2*} Non-Selective

| | |
|---|---|
| Source: | Wistar Rat cerebral cortex |
| Ligand: | 0.7 nM [³H] Prazosin |
| Vehicle: | 1% DMSO |
| Incubation Time/Temp: | 30 minutes @ 25° C. |
| Incubation Buffer: | 20 mM Hepes, 2.5 mM Tris-HCl, |
| | pH 7.4 @ 25° C. |
| Non-Specific Ligand: | 1 μM Yohimbine |
| K_{D}: | 7.8 nM* |
| B_{MAX}: | 0.36 pmole/mg Protein* |
| Specific Binding: | 80%* |
| Quantitation Method: | Radioligand Binding |
| Significance Criteria: | ≧50% of max stimulation or inhibition |

These results show that 11-piperazin-1-yldibenzo[b,f][1,4]thiazepine has lower affinity than quetiapine to the α1 and α2 adrenergic receptors.

### Example 5

### In vitro Assay of 5HT_{1A} Agonism by 11-piperazin-1-yldibenzo[b,f][1,4]thiazepine

CHO membranes (10 μg protein) expressing human 5-HT_{1A} receptors were incubated in 100 μl of 20 mM HEPES, pH 7.4 assay buffer containing 10 mM MgCl₂, 100 mM NaCl, 0.1% BSA, 20 μM GDP, 200 μg WGA-PVT beads (Amersham RPNQ0001), 200 pM GTPγ³⁵S (Perkin Elmer NEG-030H). 11-piperazin-1-yldibenzo[b,f][1,4]thiazepine was incubated with the above at 11 different concentrations varying from 10 μM to 170 pM in Packard OptiPlates with shaking for 1.5 hours at room temperature. 5-HT was used as a positive control, with an EC50 15.5 nM in the assay. One μM of 5-HT was used as maximum agonist activity (100%) for the compound efficacy determination. The plates were centrifuged to settle the beads and measured in a Parkard TopCount. Using this assay, 11-piperazin-1-yldibenzo[b,f][1,4]thiazepine is shown to be a partial agonist of 5HT_{1A} receptor with an EC50 of 310 nM and a maximum efficacy of 66% relative to 1 μM of 5-HT.

### Example 6

### Oral Bioavailability

11-piperazin-1-yldibenzo[b,f][1,4]thiazepine was administered to 3 Sprague-Dawley rats each either intravenously or orally at doses of 10 μmol/kg or 30 μmol/kg, respectively, in a sodium citrate (pH 3) formulation. Blood samples were removed from each animal at several timepoints after dosing. The blood samples were centrifuged to produce plasma. Aliquots of each plasma sample were analyzed by an HPLC method with mass spectrometric detection to measure 11-piperazin-1-yldibenzo[b,f][1,4]thiazepine. The area under the plasma concentration curves (AUC) constructed from the sample measurements following iv or po administration were used to calculate oral biovailability. The calculated oral bioavailability based on the results of this study was 11% for rat.

A similar study design (different doses for oral and iv administration) was used to calculate oral bioavailability in Beagle dogs (42%) and cynomolgus monkeys (37%). Hence, 11-piperazin-1-yldibenzo[b,f][1,4]thiazepine is shown to be orally bioavailable in three species.

Brain exposure was measured in rats. For concentrations of 11-piperazin-1-yldibenzo[b,f][1,4]thiazepine in brain, rats were dosed either po or iv (n=3 per dose route). At one hour after compound administration, blood and brain samples obtained and then processed for analysis using HPLC/MS to measure concentrations of 11-piperazin-1-yldibenzo[b,f][1,4]thiazepine. Average concentrations in rats one hour after oral dosing at 30 μmol/kg po was 658 nmol/ml plasma and 2240 nmol/g brain tissue, giving a brain/plasma exposure ratio of 3.4. A similar analysis after iv dosing measured brain:plasma concentration ratios of 4.6 demonstrating penetration of the compound into the CNS.

### Example 7

### In vivo Anxiolytic Activity of 11-piperazin-1-yldibenzo[b,f][1,4]thiazepine

Anxiolytic activity of 11-piperazin-1-yldibenzo[b,f][1,4]thiazepine was tested in rats according to the Geller-Seifter conflict test. Efficacy results are presented in Table 1 and plasma levels in satellite exposure animals at 0.25 h after administration are provided in Table 2.

Subjects: 30 Male Long Evans rats were used. Subjects weighed 350-450 g at the time of testing, and were food restricted to 85% of free feeding weight by post session feeding with approximately 15 g of standard rat chow per day. All animals had free access to water except during experimental testing. Subjects were individually housed throughout the course of the experiment under a 12 hour light/dark cycle.

Apparatus: Standard 2-lever operant chambers were used (Med Associates). The chambers were fitted with two retractable response levers and a stimulus lamp over each of the 2 levers. A pellet food dispenser delivered 45 mg food pellets, (Bio Serv) to a cup located inside of the chamber below and between the 2 response levers. A lamp at the top and back of the chamber served as houselights. The grid floors of the operant chambers were interfaced to shock generators and scramblers (Med Associates). All events in the chambers were controlled and monitored by a microprocessor.

Procedure: There were two components in the procedure: 1) unsuppressed responding components (unpunished) with 2 minutes in duration and 2) suppressed responding components (punished) with 3 minutes in duration. In unpunished components, the houselights and both stimulus lamps over the response levers were turned on, the lever on the left-hand side of the chamber extended, and a food pellet was delivered following an average of 17 responses on the lever in the chamber (range 3 to 40 responses)—a variable ratio 17 schedule (VR17). The punished components followed unpunished components, and during these, the right-hand lever was extended into the chamber, and the stimulus lamps and houselights were turned on and off at 1 second intervals, in succession, which served as a cue for this component. In the punished component, food was also available under a VR17 schedule, but in addition, electrical current (0.5 seconds duration) was delivered to the grid floor of the chamber under an independent VR17 schedule. The level of the current was adjusted for each individual subject until responding was reduced in the suppressed component to a level that was about 5-10% that of the unpunished component, and ranged from 0.2 mA to 0.75 mA. Unpunished and punished components were separated by 10 second time-out periods in which both response levers were retracted and all stimulus lamps turned off. 2-Min unpunished and 3-min unpunished components alternated until 5 of each were completed. Daily sessions always began with unpunished responding component.

Rats whose responding was most stable were chosen from a larger pool of trained rats. Several doses were tested on a given day in different subjects. Each dose, then, was tested in a different sub-set of rats. The dependent variables recorded were the rate of responding in unpunished and punished components (total responses/total time under the component), the number of shocks delivered. A selective anxiolytic effect is defined as an increase in responding in the unpunished components with relatively less or no effect on responding in unpunished components. t-Tests were used to compare mean of the control's rate of responding on vehicle day of the rats used for a specific dose to the same rats means following delivery of each dose of compound (for only the rats used within each dose). Brains, CSF and plasma were collected in a satellite group of rats that match the Geller-Seifter rats to evaluate exposure levels.

Drugs: Once animals were trained to a stable baseline for 3 consecutive days, drug testing began. Drugs were administered on Tuesdays and Fridays s.c. in a volume of 1 ml/kg. Doses of 0.3, 1, 2, 5 and 10 mg/kg were dissolved in saline and the highest stock solution was prepared, and appropriate concentration prepared by serial dilutions into saline. Diazepam was supplied in an Abbott's cocktail (10% ethanol, 40% propylene glycol, 50% water) solution in a concentration of 5 mg/ml, and was prepared by serial dilution (0.3, 1 and 3 mg/kg) into 50% concentration of Abbott's cocktail. The drug had a 15 minute pre-treatment time whereas diazepam was dosed 30 minutes prior to testing. On average 6-10 rats were for each dose of drug and 3-5 for the diazepam.

Exposure Sampling: In weight and feeding status-matched subjects, terminal plasma, whole brain and CSF samples were collected. Four rats were used for each of the 4 doses of drug, with samples obtained 15 minutes after dosing.

Statistics: Absolute rate of responding in punished and unpunished components was the endpoint measured for individual subjects, and the means reported. The % control rate of responding was calculated as the (rate of responding following drug administration/rate following vehicle administration)×100. This calculation was performed for individual subjects, and the means reported. The Student t-Test was used to compare mean control rates for a given set of rats to their corresponding rate of responding after drug administration.

Results: Results are summarized below in Tables 1 and 2. Significant increases in rates of responding in the punished component, indicative of anxiolytic activity, were noted for doses of 2, 5, and 10 mg/kg.

TABLE 1

| | Dose (mg/kg s.c.) |
|---|---|
| Tested | 1, 2, 5, 10 |
| Active | 2, 5, 10 |
| Inactive | 1 |

TABLE 2

| Dose (mg/kg s.c.) | Plasma (nM) |
|---|---|
| 2 | 4,800 |
| 5 | 12,100 |
| 10 | 19,400 |

### Example 8

### In Vitro Binding Data for Brain Serotonin 5-HT₂, D1 and D2 Receptors and in Vivo Rat and Mouse Studies

An assessment of dopamine antagonism was made. The results are as follows: the binding affinity for brain serotonin 5-HT₂ receptor was 27 K₁ nM, and for dopamine D₁ and D₂ receptors was 1489 and 234 K₁ nM, respectively. These results show that the compound of the present invention as the dihydrochloride salt interacts with a broad range of neurotransmitter receptors, however, the assay also reveals that that the compound of the present invention as the dihydrochloride salt has a higher affinity for serotonin (5-HT₂) receptors relative to dopamine (D₂) receptors in the brain. It is this combination of serotonin and dopamine receptor antagonism, with higher relative 5-HT₂ to D₂ receptor affinity that indicates that the compound of Formula I or its pharmaceutically acceptable salts would act as a potent atypical antipsychotic. J. Goldstein, Quetiapine Fumarate (Seroquel): a new atypical antipsychotic, 35(3) Drugs of Today 193-210 (1999).

However, in vivo antipsychotic activity of 11-piperazin-1-yldibenzo[b,f][1,4]thiazepine dihydrochloride was tested in mice (p.o) according to a standard apomorphine climbing mouse assay and in rats (freebase) in a D-amphetamine locomotor activity test and the compound was not found to be efficacious in these tests. The methods and procedures used can be found in Warawa et al., J. Med. Chem., 2001, 44(3), 372-389, and are incorporated herein by reference.

### Example 9

### Antidepressant Effects of 11-piperazin-1-yldibenzo[b,f][1,4]thiazepine in the Mouse Forced Swim Test: A Comparison to Quetiapine

The mouse forced swim test (FST) has been used to assess antidepressant potential of novel compounds (Porsolt; Lucki). Methods: Mice were placed in cylinders of water (25.0° C.±1.5° C.) for 6 minutes. The amount of immobility was quantified during the last 4 minutes of each session. The experimental observer was blind to the treatment conditions. 11-piperazin-1-yldibenzo[b,f][1,4]thiazepine (5.0 and 10.0 mg/kg) and quetiapine (5.0, 10.0, and 20.0 mg/kg) were measured as the base and administered sc 15 minutes prior to the behavioral session. Imipramine 45.0 mg/kg and citalopram 15.0 mg/kg were measured as the salt and administered ip 60 minutes prior to the behavioural session. Results: 11-piperazin-1-yldibenzo[b,f][1,4]thiazepine (10.0 mg/kg) had antidepressant effects in this assay comparable to imipramine. Quetiapine had no effect in the FST at the doses tested. These data suggest that 11-piperazin-1-yldibenzo[b,f][1,4]thiazepine but not quetiapine has an antidepressant profile.

### Example 10

### In vivo Antidepressant Activity of 11-piperazin-1-yldibenzo[b,f][1,4]thiazepine

Antidepressant activity of 11-piperazin-1-yldibenzo[b,f][1,4]thiazepine was assessed in mice according to the tail suspension test. As shown in the results below, 11-piperazin-1-yldibenzo[b,f][1,4]thiazepine has antidepressant activity in mice.

In a first experiment, mice (n=10/group) were treated with vehicle, 2.0, 5.0, or 10.0 mg/kg of 11-piperazin-1-yldibenzo[b,f][1,4]thiazepine s.c. 15 minutes prior to the test session. Mice were suspended from their tails for 7 minutes. During the last 5 minutes of the test, the duration of immobility was recorded. In a second experiment, mice (n=10/group) were treated with vehicle, 30 mg/kg 11-piperazin-1-yldibenzo[b,f][1,4]thiazepine, or 30 mg/kg fluoxetine p.o. 60 minutes prior to the test session. Mice were suspended from their tails for 7 minutes. During the last 5 minutes of the test, the duration of immobility was recorded.

Results of the first experiment showed that 11-piperazin-1-yldibenzo[b,f][1,4]thiazepine reduced immobility at all doses tested. The dose at 10.0 mg/kg significantly reduced immobility compared with controls, p<0.01, reducing immobility by more than ½ that of the vehicle control group.

Results of the second experiment showed significant main effect of treatment for the dose tested (F(2,27)=8.391). Both 11-piperazin-1-yldibenzo[b,f][1,4]thiazepine and fluoxetine at 30 mg/kg significantly reduced immobility compared to the vehicle control group, p's<0.01, reducing immobility by more than ½ that of the vehicle control group.

Various modifications of the invention, in addition to those described herein, will be apparent to those skilled in the art from the foregoing description. Such modifications are also intended to fall within the scope of the appended claims.

What is claimed is:

1. A method of treating at least one symptom or condition associated with an Anxiety Disorder or a Mood Disorder in a human comprising administering to the human an oral pharmaceutical composition comprising 11-piperazin-1-yldibenzo[b,f][1,4]thiazepine or its pharmaceutically acceptable salt in an amount effective to treat the symptom or condition associated with the Anxiety Disorder or Mood Disorder.

2. The method as recited in claim 1 wherein the Anxiety Disorder is selected from the group consisting of Panic Disorder Without Agoraphobia, Panic Disorder With Agoraphobia, Agoraphobia Without History of Panic Disorder, Specific Phobia, Social Phobia, Obsessive-Compulsive Disorder, Postaumatic Stress Disorder, Acute Stress Disorder, Generalized Anxiety Disorder, and Generalized Anxiety Disorder Due to a General Medical Condition, and wherein the Mood Disorder is selected from the group consisting of a) Depressive Disorders, including but not limited to Major Depressive Disorder and Dysthymic Disorder and b) Bipolar Depression and/or Bipolar mania Disorder, c) Cyclothymic Disorder and d) Mood Disorder Due to a General Medical Condition.

3. The method as recited claim 1 wherein the Anxiety Disorder is Generalized Anxiety Disorder.

4. The method as recited claim 1 wherein the Mood Disorder is selected from the group consisting of Depressive Disorder, Bipolar Depression and Bipolar mania.

5. The method as recited in claim 4 wherein the Depressive Disorder is Major Depressive Disorder.

6. The method as recited in claim 4 wherein the Mood Disorder is Bipolar Depression.

7. The method as recited in claim 4 wherein the Mood Disorder is selected from Bipolar Depression or Bipolar mania including Bipolar I Disorder, including those with manic, depressive or mixed episodes, and Bipolar II Disorder.

8. The method as recited in claim 7 where the Mood Disorder is Bipolar I Disorder including those with manic, depressive or mixed episodes.

9. The method as recited in claim 7 wherein the Mood Disorder is Bipoloar II Disorder.

10. The method as recited in claim 1 wherein the composition is a solid dosage form.

11. The method as recited in claim 1 wherein 11-piperazin-1-yldibenzo[b,f][1,4]thiazepine comprises a free base.

12. The method as recited in claim 1 wherein the amount comprises up to about 750 mg per day.

13. The method as recited in claim 12 where the amount comprises from about 75 mg to about 750 mg per day.

14. The method as recited in claim 12 wherein the amount comprises from about 1 mg to about 600 mg per day.

15. The method as recited in claim 12 wherein the amount comprises from about 100 to about 400 mg per day.

16. An oral pharmaceutical composition comprising the compound of Formula I: 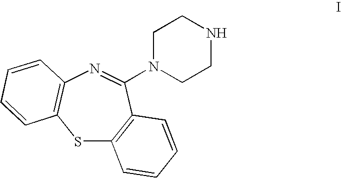
or a pharmaceutically acceptable salt thereof together with at least one pharmaceutically acceptable carrier or diluent, wherein said compound of Formula I is present in said oral composition in an effective amount for treating at least one symptom or condition associated with an Anxiety Disorder or a Mood Disorder.

17. The composition as recited in claim 16 wherein the Anxiety Disorder is selected from the group consisting of Panic Disorder Without Agoraphobia, Panic Disorder With Agoraphobia, Agoraphobia Without History of Panic Disorder, Specific Phobia, Social Phobia, Obsessive-Compulsive Disorder, Postaumatic Stress Disorder, Acute Stress Disorder, Generalized Anxiety Disorder and Generalized Anxiety Disorder Due to a General Medical Condition, and wherein the Mood Disorder is selected from the group consisting of a) Depressive Disorders, including Major Depressive Disorder and Dysthymic Disorder and b) Bipolar Depression and/or Bipolar mania, c) Cyclothymic Disorder and d) Mood Disorder Due to a General Medical Condition.

18. The composition as recited in claim 17 wherein the Disorder comprises Generalized Anxiety Disorder.

19. The composition as recited claim 17 wherein the Mood Disorder is selected from the group consisting of Depressive Disorder, Bipolar Depression and Bipolar mania.

20. The composition as recited in claim 19 wherein the Depressive Disorder is Major Depressive Disorder.

21. The composition as recited in claim 19 wherein the Mood Disorder is Bipolar Depression.

22. The composition as recited in claim 19 wherein the Mood Disorder is selected from Bipolar Depression or Bipolar mania including Bipolar I Disorder, including those with manic, depressive or mixed episodes, and Bipolar II Disorder.

23. The composition as recited in claim 22 where the Mood Disorder is selected from Bipolar I Disorder including those with manic, depressive or mixed episodes.

24. The composition as recited in claim 22 wherein the Mood Disorder is Bipoloar II Disorder.

25. The composition as recited in claim 16 wherein the 11-piperazin-1-yldibenzo[b,f][1,4]thiazepine comprises a free base.

26. The composition as recited in claim 16 wherein the composition is a solid dosage form.

27. The composition as recited in claim 16 wherein the amount is up to about 750 mg.

28. The composition as recited in claim 27 wherein the amount is about 75 mg to about 750 mg.

29. The compound as recited in claim 27 wherein the amount is from about 1 mg to about 600 mg.

30. The composition as recited in claim 27 where the amount is from about 100 to about 400 mg.
